(19) United States
(12) Patent Application Publication
(10) Pub. No.: US 2018/0231571 A1
(43) Pub. Date: Aug. 16, 2018

(54) BIOMARKER FOR DIAGNOSIS OF NEUROMYELITIS OPTICA OR MULTIPLE SCLEROSIS AND USE THEREOF

(71) Applicant: National Cancer Center, Gyeonggi-do (KR)

(72) Inventors: Ho Jin KIM, Gyeonggi-do (KR); Sunil HWANG, Charlotte, NC (US)

(21) Appl. No.: 15/696,694

(22) Filed: Sep. 6, 2017

(30) Foreign Application Priority Data
Feb. 16, 2017 (KR) 10-2017-0021231

(51) Int. Cl. G01N 33/68; G01N 33/92

(52) U.S. Cl. CPC G01N 33/6896; G01N 33/92; G01N 2333/47; G01N 2333/4713; G01N 2800/285; G01N 2800/28; G01N 2333/775; G01N 2333/78

(57) ABSTRACT

Disclosed herein is a use of glial fibrillary acidic protein (GFAP) and fibronectin as markers for distinguishing between neuromyelitis optica (NMO) and multiple sclerosis (MS). The biomarker composition of the present disclosure for distinguishing between NMO and MS enables early diagnosis of NMO or MS which are difficult to be differentiated from each other at an early stage and appropriate treatment to be applied, and can be utilized in the study of NMO and MS through proteome analysis of cerebrospinal fluid exosomes in patients.

### PRIORITY STATEMENT

This application claims priority under 35 U.S.C. § 119 to Korean Patent Application No. 10-2017-0021231, filed on Feb. 16, 2017 in the Korean Intellectual Property Office (KIPO), the contents of which are herein incorporated by reference in their entirety.

### FIELD OF TECHNOLOGY

The present disclosure relates to a marker composition for distinguishing between neuromyelitis optica (NMO) and multiple sclerosis (MS), comprising glial fibrillary acidic protein (GFAP) and fibronectin.

### BACKGROUND

Neuromyelitis optica (NMO) is a serious inflammatory disease of the central nervous system, characterized by severe, recurrent relapses leading to disability. Previously NMO had been considered as one of a clinical variant of multiple sclerosis (MS). However, the discovery of a disease-specific autoantibody against aquaporin-4 (AQP4) has dramatically changed the clinical definition of NMO and our understanding of the pathophysiology of this disease. NMO is now considered as an AQP4 antibody-mediated astrocytopathic disease distinct from MS. AQP4 antibodies are currently present in many patients with NMO and used as a marker capable of clearly differentiating NMO from MS as well as other inflammatory diseases of the central nervous system. However, the serologic status of AQP4 antibodies cannot be standardized due to their variable expression in many patients, and some patients with NMO phenotypes may be seronegative for AQP4 antibodies. Therefore, there remains a need for research on protein markers that can clearly diagnose and predict NMO and MS and monitor its therapeutic response.

Exosomes play a key role in intracellular signaling by transporting molecules through membrane vesicle trafficking. Exosomes can be a new source for biomarker discovery because they contain proteins, messenger RNAs, and microRNAs.

Cerebrospinal Fluid (CSF) is the most valuable biological sample for investigation of inflammatory central nervous system (CNS) disorders since it reflects the physical status of the central nervous system. CSF exosomes play an important role in a variety of biological processes including intracellular trafficking as well as intracellular connectivity. The changes in exosome proteomes may reflect the disease status by altering the protein subsets resulting from spinal cord lesions. Therefore, the present inventors have concluded that the properties of exosomes could help to explain the intracellular changes including chronic inflammatory diseases of the central nervous system and to understand the pathological process.

The implication of exosomes in the pathogenesis of MS has recently been known. A large number of extracellular vesicles from peripheral blood were observed more frequently in patients with MS than in healthy groups. The roles of extracellular vesicles include disruption of the blood-brain barrier, spread of inflammation in the parenchyma, and repair of demyelinating diseases. However, recent information about the biological significance of extracellular vesicles in MS, particularly information about exosomes in CSF, is limited, and there is no understanding of CSF exosomes in patients with NMO. In this regard, International Patent Application WO 2005-051178 discloses glial fibrillary acidic protein (GFAP) as a NMO-specific marker, and Am J Pathol. 1989 July; 135 (1): 161-168 discloses a marker for diagnosing MS from CNS disorders except for MS and normal condition.

### SUMMARY

As described above, the present inventors have completed this invention by confirming that glial fibrillary acidic protein (GFAP) and fibronectin can be used as biomarkers for diagnosing/distinguishing between neuromyelitis optica (NMO) and multiple sclerosis (MS).

Accordingly, the present disclosure is directed to a biomarker composition for diagnosing NMO or MS.

It is an object of the present disclosure to provide a biomarker composition for distinguishing between neuromyelitis optica (NMO) and multiple sclerosis (MS), comprising glial fibrillary acidic protein (GFAP) and fibronectin.

It is another object of the present disclosure to provide a kit for diagnosing neuromyelitis optica (NMO) or multiple sclerosis (MS), comprising glial fibrillary acidic protein (GFAP) and fibronectin.

It is still another object of the present disclosure to provide a method for distinguishing between neuromyelitis optica (NMO) and multiple sclerosis (MS) using the composition as described above.

It is still another object of the present disclosure to provide a method for detecting a biomarker of neuromyelitis optica (NMO) or multiple sclerosis (MS), comprising measuring the presence or amount of glial fibrillary acidic protein (GFAP) and fibronectin in a human biological sample to provide information necessary for diagnosing NMO or MS.

It is still another object of the present disclosure to provide a method for diagnosing neuromyelitis optica (NMO), comprising the steps of providing a biological sample from a subject in need of diagnosis of NMO; detecting a presence of glial fibrillary acidic protein and fibronectin from the sample; and diagnosing NMO if both the glial fibrillary acidic protein and the fibronectin are present in the sample.

It is still another object of the present disclosure to provide a method for diagnosing multiple sclerosis (MS), comprising the steps of providing a biological sample from a subject in need of diagnosis of MS; detecting a presence of glial fibrillary acidic protein and fibronectin from the sample; and diagnosing MS if the fibronectin is present but the glial fibrillary acidic protein is not present in the sample.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 shows the results of TEM image (A), flow cytometry (B), nanoparticle tracking analysis (C), exosome yield (D) for each group and western blot (E) using exosome-specific markers, for verifying the exosomes isolated from cerebrospinal fluid.

FIG. 2 shows results of western blots for verifying nine (9) disease-related proteins identified by proteomic analysis for their specificity to neuromyelitis optica (NMO) and multiple sclerosis (MS).

FIG. 3 shows the result of a flow cytometry (FACS) using antibodies against human glial fibrillary acidic protein (GFAP) and fibronectin to confirm expression levels of GFAP and fibronectin in the exosomes of NMO-high group and MS-high group.

### DETAILED DESCRIPTION

Hereinafter, the present invention will be described in more detail.

In order to identify diagnostic markers capable of distinguishing between neuromyelitis optica (NMO) and multiple sclerosis (MS) in cerebrospinal fluid (CSF) exosomes in differentiating manner, we have compared CSF exosomesin patients with NMO with those in patients with MS using proteomic analysis. Then, we have identified new protein substances that are expressed at significantly elevated levels in the NMO patient group compared to the MS group or are expressed at significantly elevated levels in the MS group compared to the NMO group and have verified their feasibility using immunoassay. As a result, it has been found that glial fibrillary acidic protein (GFAP), fibronectin, C4b-binding protein, haptoglobin-related protein, chitinase-3-like protein 1 and apolipoprotein B-100 can be effectively used as diagnostic markers for distinguishing between NMO and MS.

In accordance with one aspect of the present disclosure, there is provided a marker composition for distinguishing between neuromyelitis optica (NMO) and multiple sclerosis (MS), comprising glial fibrillary acidic protein (GFAP) and fibronectin.

Neuromyelitis optica (NMO) is an inflammatory disease of the central nervous system (CNS) that is difficult to be differentiated from multiple sclerosis (MS) at an early stage as it has similar symptoms to MS. However, for NMO lesions, it is known that the symptoms are more common in the spinal cord than in the brain, leading to severe paralysis of the lower limbs, vision loss, and a higher recurrence rate.

Multiple sclerosis (MS) is a chronic demyelinating disease that causes inflammatory lesions in the central nervous system consisting of the brain, spinal cord, and optic nerve. MS is caused by an autoimmune mechanism. The cause of MS is known to be activated T-lymphocytes which enter the CNS across the blood brain barrier (BBB) and damage the nerves. The lesions of MS can occur anywhere in the central nervous system, so the symptoms vary depending on the location of the lesion. If the lesion is in the optic nerve, symptoms such as vision loss and central blindness may occur; if in the cerebrum, sensory and motor disorders, cognitive dysfunction and the like may occur; if in the brainstem, diplopia and dizziness may occur; if in the cerebellum, dizziness and tremor may occur; if in the spinal cord, sensory and motor disorders and dysuria may occur.

Since NMO and MS are distinct in terms of the pathogenesis and causes and need different treatments, early diagnosis of these diseases is very important.

It is known that the glial fibrillary acidic protein (GFAP), fibronectin, C4b-binding protein, haptoglobin-related protein, chitinase-3-like protein 1 and apolipoprotein B-100 are not present or cannot be detected in the cerebrospinal fluid (CSF).

Accordingly, in accordance with one aspect of the present disclosure, there is provided a diagnostic biomarker composition capable of distinguishing between NMO and MS at an early stage.

More specifically, the composition can be used to detect or diagnose any one of NMO and MS disease groups by distinguishing between the NMO patient group and MS patient group.

In one embodiment of the present disclosure, there is provided a biomarker composition, wherein the fibronectin is overexpressed in MS as compared to NMO.

In another embodiment of the present disclosure, there is provided a biomarker composition, wherein the glial fibrillary acidic protein (GFAP) is overexpressed in NMO as compared to MS.

More preferably, there is provided a biomarker composition further comprising any of C4b-binding protein, haptoglobin-related protein, chitinase-3-like protein 1 and apolipoprotein B-100.

In one embodiment of the present disclosure, there is provided a biomarker composition, wherein the C4b-binding protein, the haptoglobin-related protein, and the apolipoprotein B-100 are specifically overexpressed in NMO as compared to MS, or the chitinase-3-like protein 1 is overexpressed in MS as compared to NMO.

In one embodiment of the present disclosure, the biomarker composition can provide proteins which have been discovered by proteomic analysis of exosomes obtained from cerebrospinal fluid (CSF) samples.

Specifically, a protein biomarker capable of diagnosing NMO or MS in a differentiating manner may be provided by isolating exosomes from cerebrospinal fluid (CSF) of NMO and MS patient groups and comparing and analyzing proteins overexpressed in CSF exosomes of the NMO and MS patient groups via exosomal proteomic analysis.

In one embodiment of the present disclosure, the biomarker composition may comprise, without limitation, any one or more markers selected from the group consisting of glial fibrillary acidic protein (GFAP), fibronectin, C4b-binding protein, haptoglobin-related protein, chitinase-3-like protein 1 and apolipoprotein B-100.

In accordance with another aspect of the present disclosure, there is provided a kit for diagnosing NMO or MS, comprising glial fibrillary acidic protein (GFAP) and fibronectin. In another way, there is provided a kit for diagnosing neuromyelitis optica (NMO) and/or multiple sclerosis (MS), comprising an agent capable of detecting glial fibrillary acidic protein (GFAP) and fibronectin, respectively. The kit may comprise an agent capable of detecting glial fibrillary acidic protein (GFAP) and an agent capable of detecting fibronectin. Alternatively, the kit may comprise an agent capable of detecting both glial fibrillary acidic protein (GFAP) and fibronectin.

More preferably, there is provided a kit further comprising any of C4b-binding protein, haptoglobin-related protein, chitinase-3-like protein 1, and apolipoprotein B-100. In another way, there is provided a kit for diagnosing neuromyelitis optica (NMO) and/or multiple sclerosis (MS), comprising an agent capable of detecting any of C4b-binding protein, haptoglobin-related protein, chitinase-3-like protein 1, and apolipoprotein B-100, respectively. For example, the agent capable of detecting biomarker proteins such as GFAP or fibronectin, etc., may be an antibody specifically binding to the biomarker proteins according to the present invention, such as, GFAP, fibronectin, C4b-binding protein, haptoglobin-related protein, chitinase-3-like protein 1, and apolipoprotein B-100.

In accordance with another aspect of the present disclosure, there is provided a method for distinguishing between NMO and MS using the glial fibrillary acidic protein (GFAP) and fibronectin.

More specifically, there is provided a method for distinguishing between NMO and MS, comprising the steps of:

obtaining a sample from a subject in need of diagnosis of NMO or MS:

measuring protein expression levels of one or more biomarkers selected from the group consisting of glial fibrillary acidic protein (GFAP) and fibronectin from the sample, respectively; and

comparing the protein expression levels of the biomarkers obtained from the subject to determine the onset of NMO or MS in the subject.

The determination step provides a method for distinguishing between NMO and MS, comprising, based on the comparison of the expression levels of the biomarkers, diagnosing the onset of NMO if the glial fibrillary acidic protein (GFAP) is overexpressed in the subject, or diagnosing the onset of the MS if the fibronectin is overexpressed in the subject.

Alternatively, there is provided a method for distinguishing between NMO and MS, comprising diagnosing NMO if both the glial fibrillary acidic protein (GFAP) and the fibronectin are expressed in the subject, or diagnosing MS if the fibronectin is expressed but not the glial fibrillary acidic protein.

More preferably, there is provided a method for distinguishing between NMO and MS, comprising the steps of:

measuring protein expression levels of biomarkers further comprising any one or more of the C4b-binding protein, the haptoglobin-related protein, the chitinase-3-like protein 1 and the apolipoprotein B-100 from the sample, respectively; and

comparing the protein expression levels of the biomarkers obtained from the subject to determine the onset of NMO or MS in the subject.

The determination step provides a method for distinguishing between NMO and MS, further comprising, based on the comparison of the expression levels of the biomarkers, diagnosing the onset of MS if the chitinase-3-like protein 1 is overexpressed in the subject, or diagnosing the onset of NMO if the C4b-binding protein, the haptoglobin-related protein or the apolipoprotein B-100 is overexpressed in the subject.

In accordance with another aspect of the present disclosure, there is provided a method for detecting a biomarker of NMO or MS, comprising measuring the presence or amount of glial fibrillary acidic protein (GFAP) and fibronectin in a human biological sample to provide information necessary for diagnosing NMO or MS.

In one embodiment of the present disclosure, there is provided a method further comprising measuring the presence or amount of any one or more of C4b-binding protein, haptoglobin-related protein, chitinase-3-like protein 1 and apolipoprotein B-100 in the human biological sample.

In accordance with another aspect of the present disclosure, there is provided a method for diagnosing neuromyelitis optica (NMO), comprising the steps of:

providing a biological sample from a subject in need of diagnosis of NMO:

detecting a presence of glial fibrillary acidic protein and fibronectin from the sample; and

diagnosing NMO if both the glial fibrillary acidic protein and the fibronectin are present in the sample.

In accordance with another aspect of the present disclosure, there is provided a method for diagnosing neuromyelitis optica (NMO), comprising the steps of:

providing a biological sample from a subject in need of diagnosis of NMO;

measuring protein expression levels of glial fibrillary acidic protein and fibronectin from a sample, and

diagnosing NMO if both the glial fibrillary acidic protein and the fibronectin are expressed.

In accordance with another aspect of the present disclosure, there is provided a method for diagnosing multiple sclerosis (MS), comprising the steps of:

providing a biological sample from a subject in need of diagnosis of MS;

detecting a presence of glial fibrillary acidic protein and fibronectin from the sample; and

diagnosing MS if the fibronectin is present but the glial fibrillary acidic protein is not present in the sample.

In accordance with another aspect of the present disclosure, there is provided a method for diagnosing multiple sclerosis (MS), comprising the steps of:

providing a biological sample from a subject in need of diagnosis of MS;

measuring protein expression levels of glial fibrillary acidic protein and fibronectin from the sample; and

diagnosing MS if the fibronectin is expressed but not glial fibrillary acidic protein.

In accordance with another aspect of the present disclosure, there is provided a method for providing information necessary for diagnosing NMO or MS, comprising the steps of:

measuring protein expression levels of glial fibrillary acidic protein and fibronectin from a sample; and

diagnosing NMO if both the glial fibrillary acidic protein and the fibronectin are expressed in the subject; or diagnosing MS if the fibronectin is expressed but not glial fibrillary acidic protein.

In one embodiment of the present disclosure, there is provided a method further comprising measuring the presence or amount of any one or more of C4b-binding protein, haptoglobin-related protein, chitinase-3-like protein 1 and apolipoprotein B-100 in the human biological sample.

More specifically, examples of the sample include, without limitation, blood, plasma, bone marrow fluid, lymph fluid, saliva, leakage fluid, amniotic fluid, cerebrospinal fluid, and the like, preferably cerebrospinal fluid.

In addition, the present disclosure provides a method for diagnosing idiopathic longitudinally extensive transverse myelitis (I-LETM), comprising identifying the expression of fibronectin, ceruloplasmin. apolipoprotein B-100, alpha-2-macroglobulin, fibrinogen beta-chain, chitinase-3-like protein, galectin-3 binding protein, C4b-binding protein alpha chain, and glial fibrillary acidic protein.

Further, the fibronectin, the ceruloplasmin, the apolipoprotein B-100, the alpha-2-macroglobulin, the fibrinogen beta-chain, the chitinase-3-like protein, the galectin-3 binding protein, the C4b-binding protein alpha chain, and the glial fibrillary acidic protein mentioned above may be biomarkers for diagnosis of idiopathic longitudinally extensive transverse myelitis, and may be constructed into a kit for identifying the expression of these proteins.

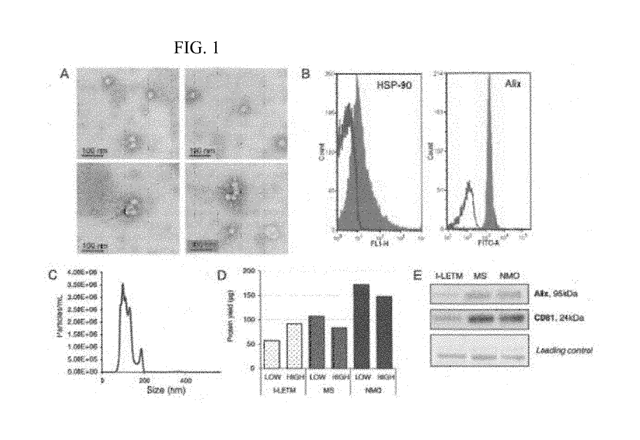

In particular, the present disclosure is based on the experimental results confirmed by Examples (e.g., FIGS. 1 to 3), as follows:
(i) glial fibrillary acidic protein may not be expressed in I-LETM and MS, but may be expressed at high levels in NMO, for example 4- to 6-fold, or about 5-fold higher as compared to I-LETM and MS;
(ii) fibronectin may be expressed at low levels in I-LETM and NMO, but may be expressed at high levels in MS, for example 2- to 4-fold, or 3-fold higher as compared to I-LETM and NMO;
(iii) ceruloplasmin may be expressed at low levels in I-LETM, and expressed at moderate levels in MS, which is at a level higher than in I-LETM, and expressed at high levels in NMO, for example about 1.5- to 3-fold higher as compared to I-LETM and MS;
(iv) apolipoprotein B-100 may be expressed at low levels in MS, and expressed at moderate levels in I-LETM and NMO, for example about 2- to 4-fold higher as compared to MS;
(v) alpha-2-macroglobulin may be expressed at low levels in I-LETM and MS, and expressed at high levels in NMO, for example 2- to 4-fold higher as compared to I-LETM and MS.
(vi) fibrinogen beta-chain may be expressed at low levels in I-LETM, and expressed at moderate levels in MS, and expressed at high levels in NMO, for example about 1.5- to 3-fold, or about 1.8-fold higher as compared to I-LETM and MS;
(vii) haptoglobin-related protein may not be expressed in I-LETM and MS, and expressed at moderate levels in NMO, for example about 4- to 6-fold, or about 5-fold higher as compared to I-LETM and MS;
(viii) C4b-binding protein alpha-chain may not be expressed in I-LETM and MS, and expressed at moderated levels in NMO, for example about 4- to 6-fold, or about 5-fold higher as compared to I-LETM and MS;
(ix) galectin-3-binding protein may be expressed at low levels in I-LETM, and expressed at moderate levels in MS, and also expressed at moderate levels in NMO, for example 1.5- to 2-fold higher as compared to I-LETM.

As described above, based on the difference in the expression level of each protein in I-LETM, MS and NMO, one or more of the proteins mentioned in (i) to (ix) may be used as a biomarker for diagnosis of one or more of the three diseases, and a method for diagnosis of the diseases using the same may be provided.

### Advantageous Effects of Invention

The biomarker composition of the present disclosure for distinguishing between neuromyelitis optica (NMO) and multiple sclerosis (MS) enables early diagnosis of NMO or MS, which are difficult to be differentiated from each other at an early stage and appropriate treatment to be applied, and can be utilized in the study of NMO and MS through proteome analysis of cerebrospinal fluid exosomes in patients.

Hereinafter, the present invention will be described in detail with reference to Examples.

### Example 1: Sample Preparation

Cerebrospinal fluid (CSF) samples were obtained from the National Cancer Center (NCC, Republic of Korea). A total of thirty-two (32) cerebrospinal fluid samples were collected with a lumbar puncture according to a standardized protocol; 10 samples from patients with neuromyelitis optica (NMO) and patients with multiple sclerosis (MS), and an additional 12 samples from patients with idiopathic longitudinally extensive transverse myelitis (I-LETM) which are negative for anti-AQP4 (anti-Aquaporin-4) and anti-MOG (anti-myelin oligodendrocyte glycoprotein). Sample collection was performed according to the experimental procedure as approved by the Institutional Review Board of the National Cancer Center, and the samples were stored in a freezer at −80°. All subjects were Asian, and most of the NMO patients were female (8 out of 10), as shown in Table 1 below, and one NMO patient was comorbid with Sjogren's syndrome, i.e., a patent suffering from both chronic diseases at the same time.

TABLE 1
Clinical information about cerebrospinal fluid samples

| | | I-LETM, | | MS, | | NMO, | |
|---|---|---|---|---|---|---|---|
| | | n = 12 | | n = 10 | | n = 10 | |
| | | Male | Female | Male | Female | Male | Female |
| Age | <30 | 1 | — | 1 | 2 | — | 2 |
| (years) | 31-40 | 6 | 1 | 2 | 4 | — | 3 |
| | 41-50 | 2 | — | — | 1 | 1 | 2 |
| | >51 | 2 | — | — | — | — | 2 |
| Ethnicity | Asian | 11 | 1 | 3 | 7 | 1 | 9 |
| Therapy | Yes | 1 | 1 | 3 | 4 | 1 | 8 |
| | No | 10 | | | 3 | 1 | — |

| | | | | |
|---|---|---|---|---|
| Diagnosis | Subject | Hyper- | Nontoxic single | Sjogren's |
| | with | thyroidism | thyroid nodule | syndrome |
| | comor- | (M, 48) | (F, 22) | (F, 38) |
| | bidity | | | |

The CSF samples were characterized by cytokine and chemokine profiling of patients and classified into two groups based on the expression levels of MCP-1 (monocyte chemoattractant protein-1) and IP-10 (interferon gamma-induced protein-10). MILLIPLEX MAP human neurodegenerative disease marker panel kit (Millipore, Billerica. Mass., USA) and Bio-Plex human cytokine (27-plex) assay kit (Bio-Rad, Hercules, Calif., USA) were used according to the manual protocol to characterize and classify the CSF samples. Thereafter, the CSF samples were obtained based on immunological classification to provide sufficient amount for exosome preparation, and then a total of six experimental groups (I-LETM-high. I-LETM-low, MS-high, MS-low, NMO-high and NMO-low) were classified.

### Example 2: Identification of Exosomal Proteins from Cerebrospinal Fluid

(1) Isolation of Exosomes

Exosome isolations were prepared using a differential centrifugation method reported previously with minor modifications. The equal amounts (500 μl) of CSFs in 5 patients per each group were pooled and then filtered through 0.45 μm PVDF membrane, followed by centrifugation at 18,000×g for 30 minutes. The supernatant was transferred to a clean tube and centrifuged overnight at 200,000×g. The pellets obtained by centrifugation were washed with PBS, followed by additional centrifugation at 200,000×g. The resulting final pellets were suspended in PBS containing 1×protease inhibitor cocktail to generate the intact exosome suspension. The isolated exosomes were fixed with 4% paraformaldehyde and then photographed with TEM. The results are shown in FIG. 1-(A). As a result of TEM, exosomes showed spherical microvesicles having a diameter of 40 nm to 60 nm.

It is known that the external diameter of exosomes varies from 60 nm to 200 nm, depending on the location of the tissue of the exosome. Nanoparticle tracking analysis (NTA) using Nano Sight N5500 (Malvern Instruments, Malvem, UK) revealed that the sizes of CSF exosomal particles isolated according to Example 1 ranged from 60 nm to 200 nm (FIG. 1-(B)).

In addition, exosome suspension was mixed with an equal volume of 2×RIPA buffer for solubilization of exosomal membrane of the exosomal portion in exosome/PBS suspension, which were separated by SDS-PAGE, and then subjected to in-gel tryptic digestion for proteome analysis.

(2) Analysis of Exosomes

1) Flow Cytometry (FACS)

To perform flow cytometry (FACS) using Alix (Programmed cell death-6 interacting protein) antibody as an exosome-specific molecule, for validation of the exosomes isolated in Example 1, 10 μg of intact exosomes were incubated overnight at 4° C. with 1 μl of aldehyde/sulfate latex bead (4 μm) suspension for coupling. Thereafter, the exosomes were washed twice with FACS buffer composed of 0.5×PBS containing 0.05% BSA, and incubated at room temperature for 1 hour with a primary antibody. Then, the exosomes were washed again with FACS buffer, incubated with a fluorescent secondary antibody for 30 minutes at room temperature, and then washed twice. After resuspension in FACS buffer, flow cytometry was performed with FACSAria II (BD Biosciences, Franklin Lakes, N.J. USA). It was found that the antibody for FACS specifically bound to the target protein, as the exosomal marker molecule was located within the vesicle, not the surface (FIG. 1-(B)). In addition, it was predicted by positive detection of Alix through flow cytometry that Alix was located on the inner surface of the endoplasmic reticulum and could interact with extra-vesicular protein.

2) Western Blot

In order to validate the exosome sample isolated in Example 1 by immunoassay, western blot was performed using Alix antibody as a representative exosome-specific molecule. After the exosomes were loaded on 10% Bis-Tris Plus gel (Life Technologies, Carlsbad, Calif., USA) and separated by electrophoresis for 30 minutes at room temperature, proteins were transferred to nitrocellulose membrane at 12 V overnight at 4° C. Then, the membrane was blocked with 5% nonfat milk buffer at room temperature for 1 hour, and incubated overnight at 4° C. with a primary antibody diluted 1:500-1:1000 in a blocking buffer. Blots were washed for 45 min with replacing TBST three times, and incubated for 1 hour at room temperature with HRP-conjugated secondary antibodies diluted 1:10000 in a blocking buffer. After washing three times with TBST for an additional 45 minutes, chemiluminescent imaging was performed using ECL prime solution, and protein expression was analyzed with a UVP Biospectrum 500 Imaging System (Upland, Calif., USA). As a result, it was found that the proteins were specifically expressed using antibodies against exosome markers, Alix and CD81 (FIG. 1-(E)).

### Example 3: Preparation for Proteome Analysis and Data Processing

30 μg of exosomal proteins were separated on 10% Bis-Tris NuPAGE gels. The proteins were stained with Coomassie Blue dye. Each gel line was cut into 10 slices, which were chopped into small pieces. The gel pieces were destained with 50% (v/v) acetonitrile (ACN) containing 25 mM ammonium bicarbonate and dehydrated with 100% ACN. After being dried in a Centrivap (Labconco, Kansas City, Mo., USA), the gel pieces were rehydrated in 50 mM ammonium bicarbonate containing 12.5 ng/μl trypsin and incubated at 37° C. for 24 hours. Peptides were extracted by adding 100 μl of 50% (v/v) acetonitrile (ACN) containing 5% (v/v) formic acid and incubating at room temperature for 30 minutes. The extracts were dried in vacuo and then suspended in 5% (v/v) acetonitrile (ACN) containing 3% (v/v) formic acid, followed by analysis using a high-resolution ion trap mass spectrometer. Peptides were separated by a reverse phase column (Nanoacquity BEHC18, 1.7 μm, 75 um×150 nm, Waters, Mass., USA) with a trap column (Nanoacquity BEHC18, 15 um, 180 um×20 nm, Waters, Mass., USA).

The human protein database from UniProtKB (20, 161 entries, date; Oct. 1, 2014) was used within Proteome Discoverer analytical software (v1.4, Thermo Scientific, Rockford, Ill., USA) applying the generic SEQUEST search algorithm. The search parameters are as follows: parent mass tolerance of 20 ppm, fragment mass tolerance of 0.8 Da (monoisotopic), variable modification on methionine of 16 Da (oxidation) and maximum missed cleavage of 2 sites using the digestion enzyme trypsin. The identified peptides were validated employing decoy database search using percolator node in Proteome Discoverer software. Exosomal proteins isolated from MS, NMO and I-LETM were subjected to proteomics analysis with duplicated analyses, the MASCOT and SEQUEST algorithms. As a result, 513 proteins were identified by the MASCOT algorithm, 473 proteins by the SEQUEST algorithm, and commonly identified proteins from both the MASCOT and SEQUEST algorithms were 442.

Spectral counts from duplicated analyses were compared using the Power Law Global Error Model (PLGEM) in order to identify the significance of the protein changes for two disease groups. Label-free quantification was performed using Proteome Discoverer and Skyline software (MacCoss Lab Software, Seattle, Wash., USA). Among the peptides generated by in-silico tryptic digestion of protein FASTA sequence, distinct peptides for each target protein were selected by BLAST search on the UniProt website. Nine (9) proteins associated with NMO and MS, which were selected by the above method, are shown in Table 2 below.

TABLE 2

| | | Raw spectral | | | | | | | | |
|---|---|---|---|---|---|---|---|---|---|---|
| | | counts | | | Average peak intensity | | | emPAI | | |
| Protein | MW | I- | | | I- | | | I- | | |
| name | (Da) | LETM | MS | NMO | LETM | MS | NMO | LETM | MS | NMO |
| glial | 50 | 0 | 0 | 50 | 0.00E+00 | 0.00E+00 | 7.11E+07 | 0.00 | 0.00 | 3.83 |
| fibrillary | | | | | | | | | | |
| acidic | | | | | | | | | | |
| protein(GF | | | | | | | | | | |
| AP) | | | | | | | | | | |
| fibronectin | 263 | 223 | 1514 | 159 | 1.60E+08 | 6.56E+08 | 1.24E+08 | 2.85 | 6.44 | 2.21 |
| ceruloplasmin | 122 | 90 | 118 | 341 | 1.09E+08 | 1.25E+08 | 1.56E+08 | 3.05 | 4.42 | 6.90 |
| apolipoprotein | 516 | 46 | 3 | 88 | 7.57E+06 | 2.84E+06 | 1.05E+07 | 0.63 | 0.04 | 0.79 |
| B-100 | | | | | | | | | | |
| alpha-2- | 163 | 170 | 207 | 725 | 8.74E+07 | 1.08E+08 | 3.05E+08 | 4.86 | 6.92 | 9.21 |
| macroglobulin | | | | | | | | | | |
| fibrinogen | 56 | 3 | 39 | 75 | 9.65E+0 | 5.61E+07 | 1.12E+08 | 0.12 | 2.99 | 6.57 |
| β-chain | | | | | | | | | | |
| haptoglobin- | 39 | 0 | 0 | 11 | 0.00E+00 | 0.00E+00 | 2.16E+07 | 0.00 | 0.00 | 0.46 |
| related | | | | | | | | | | |
| protein | | | | | | | | | | |
| C4b- | 67 | 0 | 0 | 14 | 2.42E+06 | 3.35E+06 | 6.22E+07 | 0.05 | 0.05 | 0.72 |
| binding | | | | | | | | | | |
| protein | | | | | | | | | | |
| chitinase-3- | 43 | 16 | 67 | 35 | 4.15E+07 | 2.70E+08 | 8.38E+07 | 1.27 | 3.03 | 2.22 |
| like protein 1 | | | | | | | | | | |
| galectin-3- | 65 | 8 | 21 | 23 | 2.14E+07 | 5.44E+07 | 3.84E+07 | 0.45 | 1.65 | 1.64 |
| binding | | | | | | | | | | |
| protein | | | | | | | | | | |

It was found that, among these proteins, the fibronectin and the chitinase-3-like protein 1 were overexpressed in the multiple sclerosis (MS) group, and the glial fibrillary acidic protein (GFAP) was specifically expressed in the NMO group.

In addition, alpha-2-macroglobulin, apolipoprotein B-100, ceruloplasmin, fibrinogen β-chain, galectin-3-binding protein, C4b-binding protein, haptoglobin-related protein and apolipoprotein B-100 were further identified. Among them, it was revealed that the C4b-binding protein, the haptoglobin-related protein and the apolipoprotein B-100 were NMO-specific.

### Example 4: Western Blots Using Identified Proteins

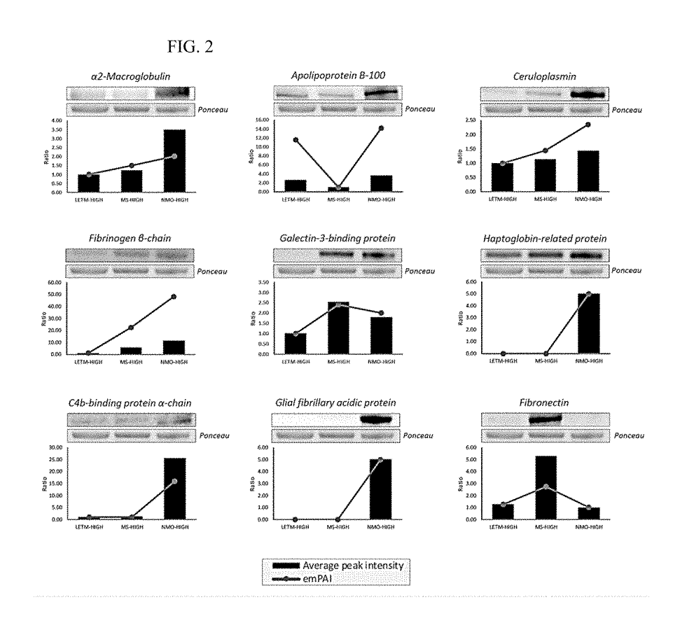

In order to verify whether nine representative disease-related proteins, obtained by statistical analysis based on spectral counts using PLGEM, were NMO- and MS-specific markers, immunoassay (western blot) and mass spectrometry based on label-free quantification were performed. The immunoblot images of the target proteins were the results calculated from the peptide ion intensities. The exponentially modified protein abundance index (emPAI) was calculated by linear correlation of the peptide ion intensities of the Western blot with relative quantification through Scaffold software (FIG. 2). As a result, it was found that GFAP, C4b-binding protein, apolipoprotein B-100 and haptoglobin-related protein were specifically expressed in NMO, and fibronectin was overexpressed in MS.

### Example 5: Flow Cytometry (FACS) of Identified Proteins

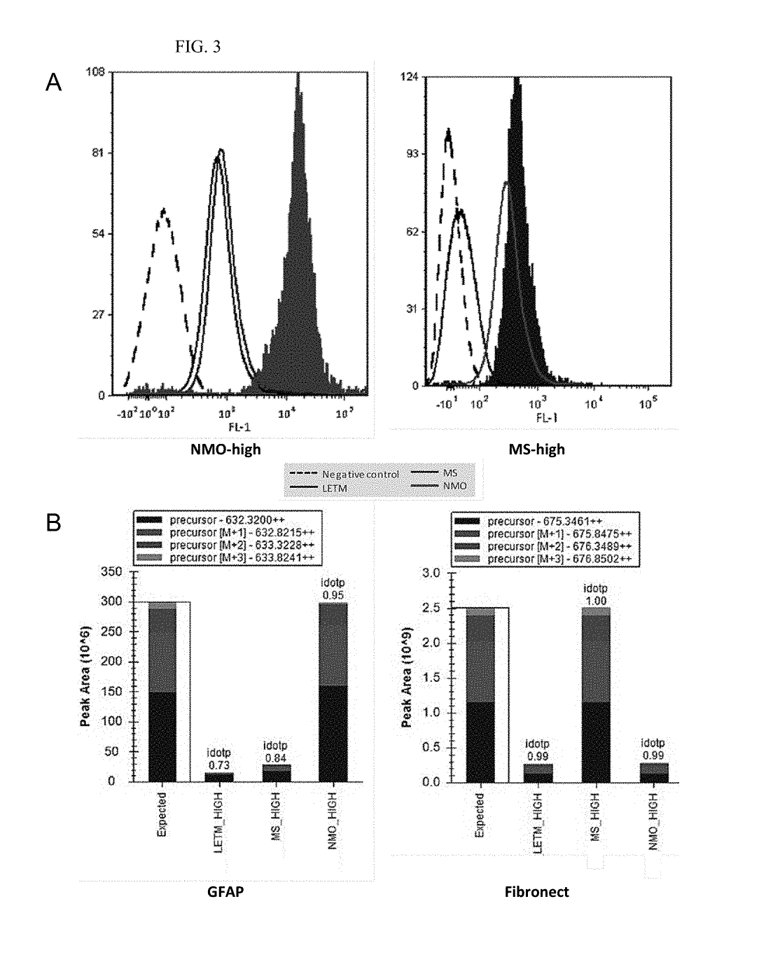

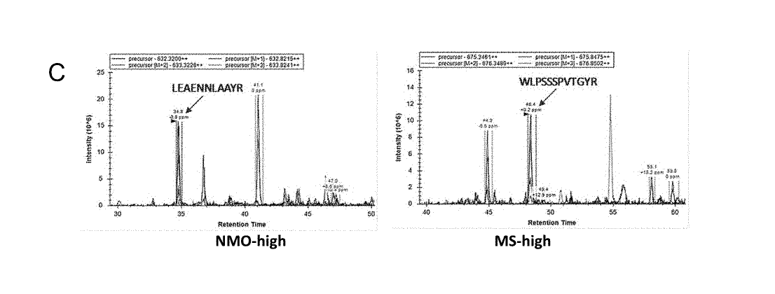

Flow cytometry (FACS) was performed for the qualitative and quantitative analysis of the proteins identified according to Example 3. The exosomal portion suspended in PBS from each disease group was subjected to flow cytometry (FACS) using antibodies against human glial fibrillary acidic protein (GFAP) and fibronectin for determination on the expression levels of the intact exosomes. As a result, as shown in FIG. 3, the histogram of the NMO-high group was significantly shifted to the right as compared with that of the MS-high or I-LETM-high group, indicating that the detection of the glial fibrillary acidic protein (GFAP) was highly increased in the NMO-high group. It was found that fibronectin was also partially detected in the exosome group derived from NMO, but was expressed at high levels in MS exosomes (FIG. 3). It was found that the polyclonal antibody against human fibronectin recognized a variety of epitopes and showed positive response in both the NMO group and the MS group, but was expressed at high levels in the MS group as in the result of immunoblots. These results were supported by label-free quantitative analysis using Skyline software including Q-Exact Hybrid Mass Spectrometric analysis and ion exchange chromatography for each of the NMO-high and MS-high groups (FIGS. 3B and 3C). Thus, it was confirmed that glial fibrillary acidic protein (GFAP) and fibronectin, which were identified by proteome analysis of cerebrospinal fluid exosomes in the NMO and MS disease groups, are an NMO-specific marker and an MS-specific marker, respectively, and thus can distinguish between NMO and MS, in conjunction with an NMO-specific molecular panel (e.g., AQP4).

Although the preferred embodiments of the present disclosure have been disclosed for illustrative purposes, those skilled in the art will appreciate that various modifications, additions and substitutions are possible, without departing from the scope and spirit of the invention as disclosed in the accompanying claims.

What is claimed is:

1. A biomarker composition for diagnosing a disease selected from the group consisting of neuronmyelitis optica (NMO) and multiple sclerosis (MS), the biomarker composition comprising:
glial fibrillary acidic protein (GFAP); and
fibronectin.

2. The biomarker composition of claim 1, wherein the fibronectin is overexpressed in the MS as compared to the NMO.

3. The biomarker composition of claim 1, wherein the GFAP is overexpressed in the NMO as compared to the MS.

4. The biomarker composition of claim 1, further comprising any of C4b-binding protein, haptoglobin-related protein, chitinase-3-like protein 1, and apolipoprotein B-100.

5. The biomarker composition of claim 4, wherein at least one of the C4b-binding protein, the haptoglobin-related protein, and the apolipoprotein B-100 is overexpressed in the NMO as compared to the MS.

6. The biomarker composition of claim 4, wherein the chitinase-3-like protein 1 is overexpressed in the MS as compared to the NMO.

7. A method for diagnosing neuromyelitis optica (NMO), comprising the steps of:
providing a biological sample from a subject in need of diagnosis of the NMO;
detecting a presence of glial fibrillary acidic protein and fibronectin from the biological sample; and
diagnosing the NMO if both the glial fibrillary acidic protein and the fibronectin are present in the sample.

8. The method of claim 7, further comprising measuring the presence or amount of any one or more of C4b-binding protein, haptoglobin-related protein, chitinase-3-like protein 1, and apolipoprotein B-100 in the biological sample.

9. The method of claim 8, wherein the biological sample is cerebrospinal fluid (CSF).

10. A method for diagnosing multiple sclerosis (MS), comprising the steps of:
providing a biological sample from a subject in need of diagnosis of the MS;
detecting a presence of glial fibrillary acidic protein and fibronectin from the biological sample; and
diagnosing the MS if the fibronectin is present but the glial fibrillary acidic protein is not present in the biological sample.

11. The method of claim 10, further comprising measuring the presence or amount of any one or more of C4b-binding protein, haptoglobin-related protein, chitinase-3-like protein 1, and apolipoprotein B-100) in the biological sample.

12. The method of claim 11, wherein the biological sample is cerebrospinal fluid (CSF).
